(19) United States
(12) Patent Application Publication
(10) Pub. No.: US 2023/0114628 A1
(43) Pub. Date: Apr. 13, 2023

(54) Prenatal Dosage Forms, Methods of Administration and Kits Thereof

(71) Applicant: ELLEMENT INC., Oakland, CA (US)

(72) Inventor: Doreen Bloch, Oakland, CA (US)

(21) Appl. No.: 17/801,989

(22) PCT Filed: Jul. 26, 2021
(86) PCT No.: PCT/US21/43140

§ 371 Date: Aug. 24, 2022

Related U.S. Application Data
(60) Provisional application No. 63/059,286, filed on Jul. 31, 2020.

(51) Int. Cl. A61K 31/295; A61K 31/30; A61K 31/375; A61K 47/55; A61K 33/04; A61K 33/18; A61K 33/30; A61K 33/32; A23L 33/00; A23L 33/16; A23L 33/29

(52) U.S. Cl. CPC A61K 31/295; A61K 31/30; A61K 31/375; A61K 47/551; A61K 33/04; A61K 33/18; A61K 33/30; A61K 33/32; A23L 33/40; A23L 33/16; A23L 33/29

(57) ABSTRACT

Disclosed herein are prenatal dosage forms formulated for different stages of the pregnancy cycle. Also disclosed are methods of administering prenatal dosage forms to a prenatal, pregnant or lactating woman. Further disclosed are kits including prenatal dosage forms.

### RELATED APPLICATIONS

This application is a National Stage of International Application No. PCT/US2021/043140, filed Jul. 26, 2021, which claims the benefit of under 35 U.S.C. § 119(e) of U.S. Provisional Patent Application No. 63/059,286 filed Jul. 31, 2020, which is incorporated by reference herein.

### FIELD

The present disclosure relates generally to prenatal dosage forms, methods of administering prenatal dosage forms and kits thereof.

### BACKGROUND

Health supplements are used by millions of people every day. Such supplements range from single-ingredient vitamins to multi-vitamins to prescription supplements incorporating hormone therapy and other medicinal treatments. Numerous of these health supplements are prepared for condition specific uses, such as anxiety or sleeplessness.

The global prenatal vitamin supplements market will reach $675M by 2025, growing rapidly in the $9B+ global maternity care market. All obstetricians in the United States recommend prenatal supplements to pregnant women. Prenatal supplements provide health benefits for the mother and the baby including a reduction in the risk and severity of autism in babies, a decrease in spina bifida and reduction in the risk of preeclampsia.

Prenatal vitamins have been developed to provide various nutrients, vitamins, minerals and other nutritional materials that are helpful to women who are pregnant, planning to become pregnant and/or lactating. The components of prenatal vitamins support the health and development of the mother and of the growing baby.

The needs of a mother and of the growing baby change throughout her pregnancy and/or whether she is preparing to become pregnant or following pregnancy during lactation. Ingestible supplements for mothers and expectant mothers are commonly available as pills formulated for once to three times daily administration. Current products typically provide only a single prenatal formulation regardless of how many weeks pregnant a mother may be and/or whether she is trying to become pregnant or is lactating post pregnancy. While available products attempt to fulfill the nutritional and health needs of a pregnant mother and her child, they do not offer optimum health benefits to the mother and her baby based on the stage (i.e., the week and/or trimester) of the pregnancy.

It is also estimated that only about 20% of the ingredients in known prenatal vitamins are actually absorbed by the mother. Conventional prenatal vitamins contain numerous compounds all of which compete for absorption. Any compounds not absorbed exit the mother's system as waste.

There is a need for methods of supplementing the nutrition of a prenatal, pregnant or lactating woman by administering to the woman different prenatal formulations at different stages of a pregnancy cycle, which may include pre-pregnancy and lactation. There is a further need for kits containing different prenatal formulations for administration during different stages of a pregnancy cycle.

### BRIEF SUMMARY

According to some embodiments, disclosed herein are methods of supplementing nutrition to a prenatal, pregnant or lactating woman, comprising, providing to the woman: one or more first dosage form, wherein the one or more first dosage form comprises at least one water-soluble compound; one or more second dosage form, wherein the one or more second dosage form comprises iron and copper; one or more third dosage form, wherein the third dosage form comprises minerals; optionally one or more fourth dosage form, wherein the dosage form comprises iodine; and one or more fifth dosage form, wherein the fifth dosage form comprises at least one fat-soluble compound, wherein the one or more first dosage form and the one or more fifth dosage form are formulated for administration at least four to twelve hours apart. In some embodiments, the at least one of the first to fifth dosage forms may be free of folate, manganese or both. In one or more embodiments, at least one of the first to fifth dosage forms comprises lutein. The at least one water-soluble compound may include one or more of vitamin B1, vitamin B2, vitamin B3, vitamin B5, vitamin B6, vitamin B12, folate, folic acid, biotin, myo-inositol or D-chiro-inositol and/or may be free of one or more of a fat-soluble compound, iodine, a mineral, iron, vitamin C or copper. The one or more second dosage form may further include vitamin C. The one or more second dosage form may be free of one or more of a water-soluble compound other than vitamin C, a fat-soluble compound, iodine or a mineral other than iron and copper. In some embodiments, the minerals may include one or more of choline, calcium, magnesium, zinc, selenium, chromium or potassium. The one or more third dosage form may be free of a water-soluble compound, a fat-soluble compound, iodine, iron and copper. According to some embodiments, the one or more fourth dosage form is free of a water-soluble compound other than iodine, a fat-soluble compound, a mineral, iron and copper. The at least one fat-soluble compound may include one or more of one vitamin A, vitamin D, vitamin E, vitamin K1, vitamin K2, eicosapentaenoic acid, docosahexaenoic acid, coenzyme Q10, lutein or zeaxanthin and may be free of one or more of a water-soluble compound, iodine, a mineral, copper, iron or vitamin C.

In some embodiments, at least one of the one or more first dosage form, one or more second dosage form, one or more third dosage form and one or more fifth dosage form are comprised in a kit and the kit is provided to the woman. According to some embodiments, at least one of the first to fifth dosage forms or one or more sixth dosage form comprises a plurality of food nutrients selected from the group consisting of choline, citrus bioflavonoid complex, ginger, apple, banana, Spirulina, marshmallow, plum, a prebiotic, a probiotic, a digestive component, an enzyme component, bromelain, kiwi, papain, alpha linolenic acid, isomalto-oligosaccharides, milk thistle extract, Silybum marianum, silymarin, mixed tocopherols, marigold extract, Tagetes erecta, sunflower lecithin, date fruit, organic Murraya keonigii, Emblic Extract, Amla, Phyllanthus emblica, Sesbania grandiflora, Sea Kelp, Psidium guajava, Guava fruit, Lemon, Annatto, Ocimum sanctum, Holy Basil leaf, Mushroom, Agaricus bisporus, Moringa oleifera, Moringa leaf, Strawberry, Cherry, Blackberry, Blueberry, Raspberry, Beet, Broccoli, Carrot, Spinach, Tomato, Green Bell Pepper, Brussels Sprout, Ginger, Garlic, Green Onion, Parsley, Cauliflower, Red Cabbage, Asparagus, Celery, Cucumber, Kale, Spirulina, Marshmallow, Plum, Bromelain, Kwiki, Papain, red raspberry, Fubius idaeus, date fruit, derivatives thereof and combinations thereof.

According to some embodiments, the one or more first dosage form is formulated for morning administration, the one or more second dosage form is formulated for morning administration, the one or more third dosage form is formulated for afternoon or evening administration, the one or more fourth dosage form is formulated for afternoon or evening administration and the one or more fifth dosage form is formulated for evening administration.

Further disclosed herein are kits, comprising: one or more first dosage form, wherein the one or more first dosage form comprises at least one water-soluble compound; one or more second dosage form, wherein the one or more second dosage form comprises iron and copper; one or more third dosage form, wherein the third dosage form comprises minerals; optionally one or more fourth dosage form, wherein the dosage form comprises iodine; one or more fifth dosage form, wherein the fifth dosage form comprises at least one fat-soluble compound; and instructions to administer the one or more first dosage form and the one or more fifth dosage form at least four to twelve hours apart.

In some embodiments, the at least one of the first to fifth dosage forms may be free of folate, manganese or both. In one or more embodiments, at least one of the first to fifth dosage forms comprises lutein. The at least one water-soluble compound may include one or more of vitamin B1, vitamin B2, vitamin B3, vitamin B5, vitamin B6, vitamin B12, folate, folic acid, biotin, myo-inositol or D-chiro-inositol and/or may be free of one or more of a fat-soluble compound, iodine, a mineral, iron, vitamin C or copper. The one or more second dosage form may further include vitamin C. The one or more second dosage form may be free of one or more of a water-soluble compound other than vitamin C, a fat-soluble compound, iodine or a mineral other than iron and copper. In some embodiments, the minerals may include one or more of choline, calcium, magnesium, zinc, selenium, chromium or potassium. The one or more third dosage form may be free of a water-soluble compound, a fat-soluble compound, iodine, iron and copper. According to some embodiments, the one or more fourth dosage form is free of a water-soluble compound other than iodine, a fat-soluble compound, a mineral, iron and copper. The at least one fat-soluble compound may include one or more of one vitamin A, vitamin D, vitamin E, vitamin K1, vitamin K2, eicosapentaenoic acid, docosahexaenoic acid, coenzyme Q10, lutein or zeaxanthin and may be free of one or more of a water-soluble compound, iodine, a mineral, copper, iron or vitamin C.

According to some embodiments, at least one of the first to fifth dosage forms or one or more sixth dosage form comprises a plurality of food nutrients selected from the group consisting of choline, citrus bioflavonoid complex, ginger, apple, banana, Spirulina, marshmallow, plum, a prebiotic, a probiotic, a digestive component, an enzyme component, bromelain, kiwi, papain, alpha linolenic acid, isomalto-oligosaccharides, milk thistle extract, Silybum marianum, silymarin, mixed tocopherols, marigold extract, Tagetes erecta, sunflower lecithin, date fruit, organic Murraya keonigii, Emblic Extract, Amla, Phyllanthus emblica, Sesbania grandiflora, Sea Kelp, Psidium guajava, Guava fruit, Lemon, Annatto, Ocimum sanctum, Holy Basil leaf, Mushroom, Agaricus bisporus, Moringa oleifera, Moringa leaf, Strawberry, Cherry, Blackberry, Blueberry, Raspberry, Beet, Broccoli, Carrot, Spinach, Tomato, Green Bell Pepper, Brussels Sprout, Ginger, Garlic, Green Onion, Parsley, Cauliflower, Red Cabbage, Asparagus, Celery, Cucumber, Kale, Spirulina, Marshmallow, Plum, Bromelain, Kwiki, Papain, red raspberry, Fubius idaeus, date fruit, derivatives thereof and combinations thereof.

According to some embodiments, the one or more first dosage form is comprised in a container for morning administration, the one or more second dosage form is comprised in the container for morning administration or in a container for afternoon administration, the one or more third dosage form is comprised in the container for afternoon administration or in a container for evening administration, the one or more fourth dosage form is comprised in the container for afternoon or in the container for evening administration and the one or more fifth dosage form is comprised in the container for evening administration.

In yet further embodiments, disclosed herein are methods of supplementing nutrition to a prenatal, pregnant or lactating woman, comprising: administering a first prenatal dosage form to the woman for about 1 week to about 12 months; administering a second prenatal dosage form to the woman for about 1 week to about 16 weeks; and administering a third prenatal dosage form to the woman for about 1 week to about 9 months, wherein the first formulation, the second formulation and third formulation are administered over consecutive time periods.

In embodiments, disclosed herein are kits, comprising: a first prenatal dosage form formulated for daily administration for a period of about 1 week to about 12 months; a second prenatal dosage form formulated for daily administration for about 1 week to about 16 weeks; and a third prenatal dosage form formulated for daily administration for about 1 week to about 9 months.

### DETAILED DESCRIPTION

Described herein are methods of supplementing the nutrition of a prenatal, pregnant or lactating woman by administering to the woman different prenatal formulations at different stages of her pregnancy cycle. There is a further need for kits containing different prenatal formulations for administration to a woman during different stages of the pregnancy cycle depending on the nutritional requirements of the woman and child.

The term “pregnancy cycle” as used herein collectively refers to the pre-pregnancy period while trying to become pregnant, the pregnancy period (i.e., the 40 weeks of pregnancy distributed over the first, second and third trimesters) and the lactation period following pregnancy during breastfeeding.

Reference throughout this specification to one embodiment, certain embodiments, one or more embodiments or an embodiment means that a particular feature, structure, material, or characteristic described in connection with the embodiment is included in at least one embodiment of the invention. Thus, the appearances of the phrases such as in one or more embodiments, in certain embodiments, in one embodiment or in an embodiment in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to a catalyst material includes a single catalyst material as well as a mixture of two or more different catalyst materials.

As used herein, the term about in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term about includes the recited number±10%, such that about 10 would include from 9 to 11.

The term at least about in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term at least about includes the recited number minus 10% and any quantity that is higher such that at least about 10 would include 9 and anything greater than 9. This term can also be expressed as about 10 or more. Similarly, the term less than about typically includes the recited number plus 10% and any quantity that is lower such that less than about 10 would include 11 and anything less than 11. This term can also be expressed as about 10 or less.

Unless otherwise indicated, all parts and percentages are by weight except that all parts and percentages of gas are by volume. Weight percent (wt. %), if not otherwise indicated, is based on the total weight of a dosage form. Volume percent (vol. %), if not otherwise indicated, is based on the total volume of the gas.

Although the disclosure herein is with reference to particular embodiments, it is to be understood that these embodiments are merely illustrative of the principles and applications of the invention. It will be apparent to those skilled in the art that various modifications and variations can be made to the compositions and methods without departing from the spirit and scope of the invention. Thus, it is intended that the invention include modifications and variations that are within the scope of the appended claims and their equivalents.

### Prenatal Dosage Forms

Prenatal dosage forms according to embodiments herein contain suitable vitamins, minerals, food nutrients and other nutritional materials for the woman and/or growing baby that are formulated for a particular stage (e.g., month, week, etc.) of a woman's pregnancy cycle. The term “vitamin” as used herein refers to an organic substance made by plants or animals. Vitamins typically can be broken down by heat, air and light. Suitable vitamins for use in prenatal formulations as described herein include, but are not limited to, vitamin A (e.g., as beta carotene from an organic food blend), vitamin C (e.g., from an organic food blend), vitamin D (e.g., D3 from lichen, D2 from organic Agaricus bisporus), vitamin E (e.g., from an organic food blend), vitamin K1, vitamin K2, thiamin (e.g., vitamin B1 from an organic food blend), riboflavin (e.g., vitamin B2 from an organic food blend), niacin (e.g., vitamin B3 from an organic food blend), vitamin B6 (e.g., from an organic food blend), folate (e.g., vitamin B9 from an organic food blend), vitamin B12 (e.g., a methylcobalamin from Saccharomyces cerevisae), biotin (e.g., vitamin B7 from an organic food blend), pantothenic acid (e.g., vitamin B5 from an organic food blend), derivatives thereof and combinations thereof. There are two different types of vitamins: water-soluble vitamins and fat-soluble vitamins. Vitamins B1-B12 and C are water-soluble whereas vitamins A, D, E and K are fat-soluble.

Vitamin A can support a growing baby's immune system, healthy skin and eyes, and also is important for alveoli development in the baby's lungs. Too much vitamin A can be harmful although larger quantities are needed later in pregnancy due to increased blood volume in the mother (e.g. in the third trimester) and even more vitamin A may be needed during breastfeeding. In embodiments, the source of vitamin A in the prenatal formulations is beta-carotene. In embodiments, a vitamin A component in prenatal dosage forms as described herein is free of synthetic and/or retinol elements. Prenatal dosage forms according to embodiments herein may include vitamin A in an amount of about 500 mcg to about 2000 mcg, about 750 mcg to about 1750 mcg, or about 800 mcg to about 1500 mcg.

Prenatal dosage forms according to embodiments herein include vitamin C in a sufficient amount to support the mother and baby. Deficiency of vitamin C can lead to high blood pressure, swelling of hands, feet and/or face (e.g., pre-eclampsia), anemia and a small baby. Vitamin C deficiency can also reduce the likelihood of placental abruption (i.e., the placenta coming away early from uterine wall) and aids in the production of collagen. A suitable level of vitamin C can also help the baby's immune system, aid in the absorption of iron and can build up storage for later use. Prenatal dosage forms according to embodiments herein may include vitamin C in an amount of about 10 mg to about 350 mg, about 25 mg to about 150 mg, or about 50 mg to about 120 mg.

Prenatal dosage forms in embodiments include vitamin D that can regulate the amount of calcium and phosphate in the body, which are needed to keep bones, teeth and muscles healthy. In embodiments, the amount of vitamin D in formulations may be higher for darker months of the year or in areas where there is less sun, or for women with darker skin. Vitamin D can reduce pre-eclampsia, postpartum hemorrhage, gestational diabetes and the low birthweight of the baby. Vitamin D deficiency is associated with higher caesarian rates. In embodiments, prenatal vitamins include vitamin D2 and are free of vitamin D3 and also include calcium as described below. Prenatal dosage forms according to embodiments herein may include vitamin D3 in an amount of about 10 mcg to about 750 mcg, about 25 mcg to about 600 mcg, or about 50 mcg to about 500 mcg.

In embodiments, prenatal dosage forms as described herein include vitamin E. Vitamin E good for fertility and may be particularly present in vitamin formulations for fertility during the pre-pregnancy stage of the pregnancy cycle. Prenatal dosage forms according to embodiments herein may include vitamin E in an amount of about 1 mg to about 30 mg, about 5 mg to about 25 mg, or about 10 mg to about 20 mg.

Prenatal dosage forms as described herein may include vitamin K1, which can help support blood clotting. Vitamin K1 may also reduce or prevent hemorrhage and anemia. Vitamin K1 may be helpful support the baby's health especially right after birth while being consumed by the breastfeeding mother. Prenatal dosage forms according to embodiments herein may include vitamin K1 in an amount of about 1 mcg to about 125 mcg, about 5 mcg to about 100 mcg, or about 10 mcg to about 75 mcg.

In embodiments, prenatal dosage forms as described herein include vitamin K2. Vitamin K2 can help with fertility in women hoping to become pregnant. Prenatal dosage forms according to embodiments herein may include vitamin K2 in an amount of about 5 mcg to about 125 mcg, about 10 mcg to about 100 mcg, or about 20 mcg to about 90 mcg.

Prenatal dosage forms as described herein may include thiamin (vitamin B1), which is helpful for development of the baby's brain. Vitamin B1 also can be lost from excess vomiting. Vitamin B1 supports the mother's nervous system, muscles and heart function. Prenatal dosage forms according to embodiments herein may include vitamin B1 in an amount of about 0.5 mg to about 15 mg, about 1 mg to about 10 mg, or about 2 mg to about 5 mg.

In embodiments, prenatal dosage forms as described herein include riboflavin (vitamin B2). Vitamin B2 can help regulates a mother's hormones. As vitamin B2 is water-soluble and can be excreted by urine, it is important to replenish this vitamin daily. Prenatal dosage forms according to embodiments herein may include vitamin B2 in an amount of about 0.5 mg to about 10 mg, about 1 mg to about 5 mg, or about 2 mg to about 3 mg.

Prenatal dosage forms as described herein may include niacin (vitamin B3). Vitamin B3 helps to regulate cholesterol and is beneficial for the brain and skin. Niacin produces nicotinamide adenine dinucleotide (NAD), which includes nicotinic acid (NA), nicotinamide (NAM), nicotinamide mononucleotide (NMN) and nicotinamide riboside (NR). NAD is a central mediator of cellular energy metabolism and a crucial link between nutritional intake, cellular metabolism and health. Suitable levels of niacin may help prevent miscarriage and birth defects. Prenatal dosage forms according to embodiments herein may include vitamin B3 in an amount of about 5 mg to about 50 mg, about 10 mg to about 40 mg, or about 12 mg to about 30 mg.

In embodiments, prenatal dosage forms as described herein include vitamin B6. Vitamin B6 can assist in brain development of the baby. It may also support nipples and can help prevent nausea. Vitamin B6 furthermore raises progesterone and supports the luteal phase to help maintain pregnancy. Prenatal dosage forms according to embodiments herein may include vitamin B6 in an amount of about 0.5 mg to about 40 mg, about 1 mg to about 30 mg, or about 2 mg to about 20 mg.

Prenatal dosage forms as described herein may include folate. Folate can decrease the risk of birth defects in the baby. For example, folate can prevent neural tube defects. Folate furthermore can create healthy new cells and promotes intelligence in children. According to embodiments, the prenatal dosage forms include folate and are free or substantially free of folic acid. The term “substantially” as used herein means undetectable or less than 1.0 wt. %, 0.1 wt. % or 0.01 wt. % of the total weight of the prenatal dosage form. Prenatal dosage forms according to embodiments herein may include folate in an amount of about 100 mcg to about 1500 mcg, about 250 mcg to about 1200 mcg, or about 500 mcg to about 800 mcg. According to embodiments, certain prenatal dosage forms, for example, for use during weeks 10 to 40 of pregnancy, may be free of folate.

In embodiments, prenatal dosage forms as described herein include vitamin B12. Cyanocobalamin is a synthetic form of vitamin B12 found only in supplements, while methylcobalamin is a naturally occurring form present in food sources and/or supplements. In embodiments, prenatal vitamins according to embodiments herein contain one or both of cyanocobalamin and methylcobalamin. Vitamin B12 can improve mood and energy. During breastfeeding and lactation, the baby gets vitamin B12 from the mother. Babies exclusively breastfed may be deficient in vitamin B12. Excess vitamin B12 is excreted in the urine so excess vitamin B12 can be consumed (e.g., more than 2.5 mcg). Many vegans/vegetarians are deficient in vitamin B12 and may require higher doses in the prenatal dosage forms. Prenatal dosage forms according to embodiments herein may include vitamin B12 in an amount of about 1 mcg to about 100 mcg, about 2.5 mcg to about 75 mcg, or about 5 mcg to about 50 mcg.

Prenatal dosage forms as described herein may include biotin (vitamin B7). Biotin can improve hair and nail growth. Biotin may also help prevent cradle cap in babies. Because biotin is water-soluble, any excess in the body may be excreted through urine. Prenatal dosage forms according to embodiments herein may include biotin in an amount of about 5 mcg to about 100 mcg, about 15 mcg to about 75 mcg, or about 30 mcg to about 50 mcg.

In embodiments, prenatal dosage forms as described herein include pantothenic acid (vitamin B5). Vitamin B5 can help with the formation of blood cells and may ease leg cramps. Vitamin B5 can also help heal skin. Large doses of vitamin B5 (e.g., in excess of about 10 g per day to about 20 g per day) can increase bleeding risk. In embodiments, prenatal vitamins as disclosed herein include both vitamin B5 and Vitamin C to assist with absorption. Prenatal dosage forms according to embodiments herein may include vitamin B5 in an amount of about 1 mg to about 50 mg, about 5 mg to about 20 mg, or about 10 mg to about 15 mg.

Unlike vitamins, minerals are inorganic compounds. Minerals maintain their chemical structure regardless of whether they are exposed to air, heat, or other elements. Suitable minerals for Prenatal dosage forms as described herein include, but are not limited to, calcium, iron, iodine, magnesium, zinc, selenium, manganese, chromium, molybdenum, copper, boron, potassium, derivatives thereof and combinations thereof. There are two types of minerals: major minerals and trace minerals. Major minerals include calcium, potassium, phosphorous, sodium, chloride, magnesium and sulfur. Trace minerals include iron, zinc, iodine, chromium, selenium, fluoride, molybdenum, copper and manganese.

In embodiments, prenatal dosage forms as described herein may include calcium. Calcium can improve polycystic ovary syndrome (PCOS)-associated hyperandrogenemia and anovulation. Calcium additionally can support embryo growth and to alkalize the body to support sperm. Calcium is also important during breastfeeding as some woman may lose bone density. Prenatal dosage forms can include vitamin D and magnesium together with the calcium to aid in absorption of calcium. Prenatal dosage forms according to embodiments herein may include calcium in an amount of about 100 mg to about 1000 mg, about 250 mg to about 750 mg, or about 500 mg to about 600 mg.

Prenatal dosage forms as described herein can include iron. Iron deficiency has been linked to infertility, miscarriage, low birth weight and preterm labor. A woman's body uses iron to make extra blood (hemoglobin) for the woman and the baby during pregnancy. Iron also helps move oxygen from a woman's lungs to the rest of the body and the baby's body. Getting enough iron can prevent iron deficiency anemia where too few red blood cells can make a woman feel tired. Calcium can inhibit iron absorption so in embodiments, the amounts of calcium and iron are suitably balanced. Iron is one of the most common nutrient deficiencies in pregnant women. There are two types of iron: heme iron and non-heme iron. Heme iron, typically found in animal sources, is a more easily absorbed form of iron than non-heme iron. Non-heme iron, which comes from plant-based sources, is not as easily absorbed as heme iron. In embodiments, prenatal dosage forms may include one or both types of iron. Although heme iron is more absorbable, research has shown more potential benefit from non-heme iron for improving ovulation. The amount of iron present in prenatal dosage forms according to embodiments herein remains low enough to prevent excessive iron in a woman's blood, which can lead to gestational diabetes, preeclampsia and/or miscarriage and also can cause constipation. Prenatal dosage forms according to embodiments herein may include iron in an amount of about 10 mg to about 75 mg, about 15 mg to about 50 mg, or about 20 mg to about 30 mg.

In embodiments, prenatal dosage forms as described herein may include iodine. Iodine can regulate metabolism and is helpful for fertility. Taking iodine is important throughout pregnancy, although both too much and too little can cause thyroid issues. Prenatal dosage forms according to embodiments herein may include iodine in an amount of about 50 mcg to about 500 mcg, about 100 mcg to about 350 mcg, or about 150 mcg to about 290 mcg. It should be noted that for those with an underactive thyroid, too much iodine can cause or worsen their condition. In some embodiments, the iodine may be formulated separately, in its own dosage form, so that subjects can omit the iodine if medically needed or personally desired.

Prenatal dosage forms as described herein can include magnesium. Magnesium is an essential mineral for both health and fertility. Magnesium is responsible for 700-800 enzyme systems in a woman's body. Magnesium supports chemical reactions in the body, helps detoxify fertility muggers within the liver and is responsible for converting food eaten by the woman into cellular energy. Magnesium furthermore can assist with sex hormone production and ovulation. Too little magnesium can lead to trouble sleeping and pre-eclampsia, so daily administration is helpful. Too much magnesium (e.g., 350+ mg per day) can cause nausea, vomiting and diarrhea. Magnesium may help with an aversion to breastfeeding, in managing stress and may assist in achieving homeostasis. Women who supplement with sufficient magnesium may have fewer pregnancy complications. In embodiments, prenatal dosage forms according to embodiments herein may contain at least one of magnesium malate or magnesium citrate, which are better absorbed by the body than magnesium aspartate. In embodiments, prenatal dosage forms as described herein are free of magnesium aspartate. Prenatal dosage forms according to embodiments herein may include magnesium in an amount of about 100 mg to about 600 mg, about 250 mg to about 450 mg, or about 320 mg to about 390 mg.

In embodiments, prenatal dosage forms as described herein may include zinc. Zinc can support fertility by regulating normal hormone function, cell division and ovulation. Zinc deficiency has been associated with complications of pregnancy and delivery, such as hypertension, premature rupture of membranes, placental abruption, prolonged labor, hemorrhage, infections, intrauterine growth retardation, low birth weight and congenital anomalies. Human bodies do not store zinc. Prenatal dosage forms according to embodiments herein may include higher levels of zinc in formulations for the second trimester than in formulations for the first trimester and may include higher levels of zinc in formulations for the third trimester than in formulations for the second trimester. Supplementing with zinc can affect the absorption of iron and copper (and to avoid deficiency), so iron and copper may be included in prenatal dosage forms as described herein to offset any losses due to zinc supplementation. Prenatal dosage forms according to embodiments herein may include zinc in an amount of about 1 mg to about 60 mg, about 5 mg to about 50 mg, or about 10 mg to about 40 mg.

Prenatal dosage forms as described herein can include selenium. Research shows that selenium can promote healthy follicles in the ovaries, which develop and release the eggs. Selenium is an antioxidant that can protect against birth defects and miscarriages caused by DNA damage. Prenatal dosage forms particularly formulated for enhanced fertility pre-pregnancy should not exceed more than 400 mcg of selenium a day because selenium toxicity (i.e., selenosis) can cause hair loss, skin rashes, nausea, diarrhea, fatigue and mood changes. Selenium is an important defense mechanism against disease as a result of its involvement in the regulation of your immune system and thyroid function. Some studies have shown that selenium supplementation during pregnancy decreased the risk of thyroid function abnormalities for women after pregnancy. Selenium can also reduce pre-term birth, pre-eclampsia and intrauterine growth restriction (IUGR). Selenium supports hormone metabolism and immune function. Too much selenium (i.e., 400 mcg+) can be toxic. In embodiments, the prenatal dosage forms may contain organically bound selenium such as high-selenium yeast. Prenatal dosage forms according to embodiments herein may include selenium in an amount of about 25 mcg to about 250 mcg, about 50 mcg to about 150 mcg, or about 75 mcg to about 100 mcg.

In embodiments, prenatal dosage forms as described herein may include manganese. Too much manganese (e.g., greater than 9 mg/day) can lead to a higher chance of pre-term labor and can cause problems in early development of the baby. Manganese can support healthy bones and cartilage and may aid in wound healing. Intestinal absorption of manganese is increased during iron deficiency, and increased iron stores (i.e., ferritin levels) are associated with decreased manganese absorption. Prenatal dosage forms according to embodiments herein may include manganese in an amount of about 0 mg to about 11 mg, about 5 mg to about 10 mg, or about 6 mg to about 8 mg. Although manganese supplementation can be helpful, as discussed above there is the possibility of complications. In some embodiments, prenatal dosage forms are free of manganese.

Prenatal dosage forms as described herein can include chromium. Chromium may enhance insulin levels and reduce appetite. Trace amounts of chromium is needed by pregnant women daily and also may assist with polycystic ovary syndrome (PCOS). Chromium can help prevent gestational diabetes and has been shown to be effective in improving glucose and insulin metabolism in women with gestational diabetes. Iron may impact absorption of chromium. Prenatal dosage forms according to embodiments herein may include iodine in an amount of about 5 mcg to about 60 mcg, about 10 mcg to about 50 mcg, or about 20 mcg to about 40 mcg.

In embodiments, prenatal dosage forms as described herein may include molybdenum. Molybdenum is an essential mineral found in high concentrations in legumes, grains and organ meats. Molybdenum activates enzymes that help break down harmful sulfites and prevents toxins from building up in the body. Molybdenum supplementation is usually unnecessary as deficiency is rare. In embodiments, prenatal dosage forms as described herein are free of molybdenum. In other embodiments, prenatal dosage forms according to embodiments herein may include molybdenum in an amount of about 0.1 mg to about 2 mg, about 0.5 mg to about 1.5 mg, or about 0.75 mg to about 1.25 mg.

Prenatal dosage forms as described herein can include copper. Copper has many benefits: it strengthens blood vessels, bones, tendons and nerves. Copper can help maintain fertility, ensures healthy pigmentation of hair and skin and promotes blood clotting. In pregnancy, excess copper levels can be associated with intrauterine growth restriction, preeclampsia and neurological disease. Taking copper with iron is important and zinc may interfere with copper absorption. Prenatal dosage forms according to embodiments herein may include molybdenum in an amount of about 0.1 mg to about 10 mg, about 0.25 mg to about 5 mg, or about 0.5 mg to about 1 mg.

In embodiments, prenatal dosage forms as described herein may include boron. Boron helps with magnesium absorption.

Prenatal dosage forms as described herein can include potassium. A low potassium level during the first half of pregnancy is associated with a lower risk for the development of gestational diabetes mellitus and severe pre-eclampsia. Potassium deficiency, which is common, can lead to leg cramps. Potassium is helpful for breastfeeding moms when need increases. Prenatal dosage forms as described herein can include about 1 mg to about 100 mg, or about 10 mg to about 99.9 mg, or about 25 mg to about 99 mg, or about 50 mg to about 99 mg, or about 75 mg to about 99 mg of potassium. Works with potassium for proper fluid balance; High potassium levels are extremely dangerous during pregnancy, it can cause a strain of a condition known as hyperkalaemia and in some cases, which can lead to kidney failure or cardiac arrest. It can also cause severe and near-fatal dehydration and aggravate a type of type 1 diabetes of the mellitus strain. Prenatal dosage forms according to embodiments herein may include potassium in an amount of about 10 mg to about 200 mg, about 25 mg to about 150 mg, or about 50 mg to about 99 mg.

Prenatal dosage forms as described herein can further include food nutrients that are beneficial to women pre-pregnancy, while pregnant and during lactation. Suitable food nutrients for Prenatal dosage forms as described herein include, but are not limited to, choline (e.g., choline bitartrate), citrus bioflavonoid complex, a tummy soothe blend of ginger, apple and banana, a prenatal wellness blend of ginger, Spirulina, marshmallow and plum, a prebiotic, a probiotic, a digestive blend/enzyme blend of bromelain, kiwi and papain, at least one omega-3 fatty acid (e.g., alpha linolenic acid, eicosapentaenoic acid, docosahexaenoic acid), isomalto-oligosaccharides, milk thistle extract (e.g., Silybum marianum, seeds, 80 wt. % silymarin), mixed tocopherols, lutein (e.g., from marigold extract, Tagetes erecta, flower), zeaxanthin (e.g., from marigold extract, Tagetes erecta, flower), sunflower lecithin, date fruit, organic Murraya keonigii (e.g., from curry leaf), Organic Emblic (e.g., Amla, Phyllanthus emblica) Extract, Organic Sesbania grandiflora (e.g., leaf), Organic Sea Kelp, Organic Psidium guajava (e.g., Guava fruit and leaf), Organic Apple (e.g., fruit), Organic Lemon (e.g., peel), Organic Annatto (e.g., fruit and seed), Organic Ocimum sanctum (e.g., Holy Basil leaf), Organic Mushroom (e.g., Agaricus bisporus), Organic Moringa oleifera (e.g., Moringa leaf), Organic Strawberry (e.g., fruit), Organic Cherry (e.g., fruit), Organic Blackberry (e.g., fruit), Organic Blueberry (e.g., fruit), Organic Raspberry (e.g., fruit), Organic Beet (e.g., root), Organic Broccoli (e.g., stalk and flower), Organic Carrot (e.g., root), Organic Spinach (e.g., leaf), Organic Tomato (e.g., fruit), Organic Green Bell Pepper (e.g., fruit), Organic Brussels Sprout (e.g., leaf), Organic Ginger (e.g., root), Organic Garlic (e.g., bulb), Organic Green Onion (e.g., bulb), Organic Parsley (e.g., leaf), Organic Cauliflower (e.g., flower and stem), Organic Red Cabbage (e.g., leaf), Organic Asparagus (e.g., flower and stem), Organic Celery (e.g., stalk), Organic Cucumber (e.g., gourd), Organic Kale (e.g., leaf), Banana, Spirulina, Marshmallow, Plum, Bromelain, Kwiki, Papain, derivatives thereof and combinations thereof.

Prenatal dosage forms as described herein can include choline. Choline (e.g., as choline bitartrate) can assist with brain development of the baby, neural tube development, neurotransmitter function, cell membranes and synapse formation. Choline also can improve the baby's memory and cognition. Choline works in concert with lutein and docosahexaenoic acid (DHA) in nature. Choline is present in eggs which some people especially vegan/vegetarians may not eat. Choline is excreted into breastmilk, so prenatal dosage forms as described herein may include a high level of choline for administration during the breastfeeding stage as compared to the pre-pregnancy and pregnancy stages. Choline levels in the woman's body are also likely to dip during the second trimester. Too much choline may lead to low blood pressure, a fishy odor and/or sweating. Prenatal dosage forms according to embodiments herein may include choline in an amount of about 100 mg to about 750 mg, about 200 mg to about 500 mg, or about 300 mg to about 400 mg.

In embodiments, prenatal dosage forms as described herein may include a citrus bioflavonoid complex. A citrus bioflavonoid complex is comprised of antioxidants. Bioflavonoids, however, can cause genetic damage, which may explain the suspected link between bioflavonoids and leukemia among infants and children. Pregnant women should not take megadoses of bioflavonoids in supplements. Prenatal dosage forms according to embodiments herein may include a citrus bioflavonoid complex in an amount of about 100 mg to about 2000 mg, about 250 mg to about 1500 mg, or about 500 mg to about 1000 mg.

Prenatal dosage forms as described herein can include a prebiotic and/or a probiotic digestive blend/enzyme blend. The prebiotic, probiotic and enzymes generally do not transfer to breastmilk, so they may be safely taken during the lactation/breastfeeding stage.

In embodiments, prenatal dosage forms as described herein may include at least one omega-3 fatty acid or a blend of omega-3 fatty acids such as alpha linolenic acid (ALA), eicosapentaenoic acid (EPA) and docosahexaenoic acid (DHA). Omega-3 fatty acids can improve ovulation and egg quality. However, too much can cause bleeding and diarrhea. Omega-3 fatty acids assist in the baby's brain development and eye and immune system development. In embodiments, omega-3 fatty acid components included in prenatal dosage forms are free of Vitamin A. In embodiments, prenatal dosage forms as described herein may include an algae (vegan) source of DHA. Many women do not get enough seafood because of dietary restrictions, so supplementing omega-3 fatty acids is very important. Prenatal dosage forms according to embodiments herein may include ALA in an amount of about 10 mg to about 500 mg, about 25 mg to about 250 mg, or about 50 mg to about 100 mg. Prenatal dosage forms according to embodiments herein may include EPA in an amount of about 25 mg to about 500 mg, about 50 mg to about 250 mg, or about 100 mg to about 150 mg. Prenatal dosage forms according to embodiments herein may include DHA in an amount of about 25 mg to about 500 mg, about 50 mg to about 350 mg, or about 100 mg to about 250 mg.

Prenatal dosage forms as described herein can include red raspberry (e.g., Rubus idaeus, leaf). Red raspberry supports uterus health and may decrease the time in labor. Red raspberry may also minimize nausea. Prenatal dosage forms according to embodiments herein may include red raspberry in an amount of about 25 mg to about 500 mg, about 50 mg to about 400 mg, or about 100 mg to about 300 mg.

In embodiments, prenatal dosage forms as described herein may include milk thistle extract (e.g., Silybum marianum, seeds, 80 wt. % silymarin). Milk thistle extract can assist in liver health. Milk thistle extract can also prevent or reduce morning sickness.

Prenatal dosage forms as described herein can include mixed tocopherols. Vitamin E together with mixed tocopherols can support cellular respiration of muscles, for example, cardiac and skeletal muscles.

In embodiments, prenatal dosage forms as described herein may include lutein (e.g., from marigold extract, Tagetes erecta, flower). Lutein can improve behavior, cognitive function and memory. Lutein also helps protect fatty acids, like DHA, from oxidative damage. In one study, infants supplemented with lutein immediately after birth demonstrated reduced development of harmful oxidizing substances known as free radicals. Lutein furthermore can support vision and nervous system development. Lutein works together with zeaxanthin (discussed below) and is present in breastmilk. Prenatal dosage forms according to embodiments herein may include lutein in an amount of about 1 mg to about 20 mg, about 2.5 mg to about 15 mg, or about 5 mg to about 8 mg.

Prenatal dosage forms as described herein can include zeaxanthin (e.g., from marigold extract, Tagetes erecta, flower). Zeaxanthin is helpful for placenta and umbilical cord development and works together with lutein. Prenatal dosage forms according to embodiments herein may include zeaxanthin in an amount of about 0.1 mg to about 7.5 mg, about 0.5 mg to about 5 mg, or about 1 mg to about 2 mg.

In embodiments, prenatal dosage forms as described herein may include date fruit. According to embodiments, date fruit may be present in prenatal dosage forms as described herein in higher levels for formulations for the third trimester than for the first and second trimesters.

Prenatal dosage forms as described herein can further include other nutritional materials that are beneficial to women pre-pregnancy, while pregnant and during lactation. Suitable other nutritional materials can include, but are not limited to, myo-inositol, potassium, betaine HCl, L-Camosine, coenzyme Q10, taurine, derivatives thereof and combinations thereof.

In embodiments, prenatal dosage forms as described herein may include myo-inositol. Inositol, or more precisely myo-inositol, is a carbocyclic sugar that is abundant in brain and other mammalian tissues. Myo-inositol mediates cell signal transduction in response to a variety of hormones, neurotransmitters and growth factors and participates in osmoregulation. It has been suggested that insulin resistance in women with PCOS can be attributed to a deficiency of myo-inositol's intracellular metabolites, D-chiro-inositol (DCI) and inositol-phosphoglycan (IPG) and mediators of insulin action. Myo-inositol also can reduce the occurrence of gestational diabetes and may help boost mood. In some women, myo-inositol can increase nausea or fatigue and can impact zinc, calcium and iron absorption.

Prenatal dosage forms as described herein can further include betaine HCl. Betaine HCl aids in protein digestion and with low potassium levels.

In embodiments, prenatal dosage forms as described herein may include L-carnosine. L-carnosine is an amino acid that can minimize the risk of autism. L-carnosine can also improve fertility.

Prenatal dosage forms as described herein can further include coenzyme Q10. Coenzyme Q10 supports fertility and reduces the risk of pre-eclampsia. In embodiments, the prenatal dosage forms as described herein may include coenzyme Q10 in the form of ubiquinol.

In embodiments, prenatal dosage forms as described herein may include taurine. An adequate supply of taurine during fetal life is important for normal beta-cell development and insulin action. An altered availability of taurine may program glucose metabolism in utero and result in type 2 diabetes in adult age. During pregnancy, taurine accumulates in the maternal tissues, to be released in the perinatal period to the fetus via the placenta and to the newborn via the maternal milk. Taurine is accumulated especially in the fetal and neonatal brain. The body makes taurine naturally, but excess taurine is excreted via urine, so supplementing with taurine can be beneficial for women during pregnancy.

Prenatal dosage forms according to embodiments herein can further include one or more pharmaceutically acceptable excipients. Examples of pharmaceutically acceptable excipients are described in the Handbook of Pharmaceutical Excipients, American Pharmaceutical Association (2012), which is incorporated by reference herein in its entirety. Suitable excipients include, but are not limited to, colorants, lubricants, thermal lubricants, antioxidants, disintegrants, binding agents, diluents, glidants, anti-adherants, chelating agents, sweeteners, flavorants, surfactants, solubilizers, stabilizers, hydrophilic polymers, hydrophobic polymers, waxes, lipophilic materials, absorption enhancers, preservatives, cross-linking agents, bioadhesive polymers, pore formers and/or combinations thereof.

Examples of suitable binding agents include, but are not limited to, cellulosic polymers (e.g., hydroxypropylmethylcellulose, hydroxypropylcellulose, hydroxyethylcellulose, etc.), polyethylene glycol, an acrylic polymer, an acrylic copolymer, a graft copolymer of polyvinyl alcohol and polyethylene glycol, a polyvinyl alcohol, alginic acid, sodium alginate, starch, pregelatinized starch, sucrose, guar gum, salts thereof, derivatives thereof and combinations thereof. Additional binders include, but are not limited to, natural or synthetic waxes, fatty alcohols (e.g., lauryl, myristyl, stearyl, cetyl or cetostearyl alcohol), fatty acids, including, but not limited to, fatty acid esters, fatty acid glycerides (e.g., mono-, di-, and tri-glycerides), hydrogenated fats, hydrocarbons, stearic acid, hydrophobic and hydrophilic materials having hydrocarbon backbones, acacia, tragacanth, sucrose, gelatin, glucose, cellulose materials (e.g., methylcellulose and sodium carboxymethylcellulose (e.g., Tylose™)), magnesium aluminum silicate, polysaccharide acids, bentonites, polyvinylpyrrolidone (povidone), polymethacrylates, and/or pregelatinized starch (such as National™ 1511 and Starch 1500). Suitable waxes include, for example, beeswax, glycowax, castor wax, camauba wax and/or other wax-like substances. A “wax-like” substance is defined as any material which is normally solid at room temperature and has a melting point of from about 30° C. to about 100° C.

Additional examples of binders which may be used include, but are not limited to, digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, natural and synthetic waxes and/or polyalkylene glycols. In certain embodiments, hydrocarbons having a melting point of between 25° C. and 90° C. may be included. Of the long-chain hydrocarbon binder materials, fatty (aliphatic) alcohols can be incorporated into the mixture according to certain embodiments. In further embodiments, the mixture or pharmaceutical composition may contain up to 80% (by weight) of at least one digestible, long chain hydrocarbon.

Examples of suitable disintegrants include, but are not limited to, sodium starch glycolate, clays (such as Veegum™ HV), celluloses (such as purified cellulose, methylcellulose, sodium carboxymethylcellulose, and carboxymethylcellulose), cross-linked sodium carboxymethylcellulose, starch, cross-linked polyvinylpyrrolidone (e.g., crospovidone), alginates, corn starches and pre-gelatinized corn starches (such as National™ 1551 and National™ 1550), gums (such as agar, guar, locust bean, pectin, and tragacanth) and/or mixtures thereof. Disintegrants can be added at any suitable step during the preparation of the pharmaceutical compositions, such as prior to granulation or during a lubrication step prior to compression or encapsulation. The pharmaceutical compositions as described herein can include one or more disintegrants in the range of about 0.5% to about 30%, or about 1% to about 10%, or about 2% to about 6%, of the total weight of the formulation.

In embodiments, the prenatal dosage form can include a glidant. A glidant is an excipient that improves the flow characteristics of a compressible powder such as tablet ingredients and/or granules. Suitable glidants include, but are not limited to, silicon dioxide, colloidal silicon dioxide and/or combinations thereof.

Suitable diluents useful in prenatal dosage forms as described herein include, but are not limited to, lactose (e.g., lactose (anhydrous), lactose (spray dried), lactose monohydrate), starch (e.g., directly compressible starch), mannitol, sorbitol, dextrose monohydrate, microcrystalline cellulose, dibasic calcium phosphate dihydrate, sucrose-based diluents, confectioner's sugar, monobasic calcium sulfate monohydrate, calcium sulfate dihydrate, calcium lactate trihydrate granular, dextrates (e.g., Emdex™), dextrose (e.g., Cerelose™) inositol, hydrolyzed cereal solids such as the Maltrons™ and Mor-Rex™, amylose, powdered cellulose (e.g., Elcema™), calcium carbonate, glycine, bentonite, polyvinylpyrrolidone, and/or combinations thereof. In certain embodiments, the pharmaceutical compositions described herein can include the diluents in the range of about 5% to about 99%, or from about 25% to about 90%, or from about 40% to about 80%, of the total weight of the formulation. Lactose has a melting point of about 202° C. Microcrystalline cellulose has a burning point of over 200° C. before it reaches a melting point, and is suitable as it does not have a low melting point.

Suitable lubricants include, but are not limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, silica, fumed silica, colloidal silica, calcium stearate, long chain fatty alcohols, boric acid, sodium benzoate and sodium acetate, sodium chloride, DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and/or combinations thereof.

Suitable anti-adherents include, but are not limited to, talc, cornstarch, colloidal silicone dioxide (Cab-O-Sil™), DL-Leucine, sodium lauryl sulfate and/or metallic stearates. In certain embodiments, the pharmaceutical compositions can include an anti-adherent in an amount from about 0.1% to about 15%, or from about 0.25% to about 10%, or from about 0.5% to about 5%, of the total weight of the formulation. Colloidal silicon dioxide is an anti-adherent agent suitable for use in some embodiments of the pharmaceutical compositions in an amount from about 0.1% to about 10%, or from about 0.25% to about 5%, or from about 0.5% to about 2%, of the total weight of the formulation. Colloidal silicon dioxide has a melting point of about 1700° C.

Other excipients (such as colorants, flavorants and sweeteners) can be utilized in embodiments of the prenatal dosage forms where they impart little to no deleterious effect on the stability of the dosage forms.

According to embodiments, prenatal dosage forms contain at least one of a colorant, opacifier, flavorant, sweetener, preservative, embrittlement inhibiting agent and/or disintegrant. Suitable colorants include, but are not limited to, azo dye, quinophthalone dye, triphenylmethane dye, xanthene dye, iron oxide, iron hydroxide, titanium dioxide, sunset yellow, allura red, amaranth, koki neil red, azogeranin, tartrazine, brilliant black, canthaxanthin, patent blue, fast green, brilliant blue, acid green, erythrosine, quinoline yellow, indigotin, curcumin, carbon black and/or combinations thereof. In embodiments, the opacifier contains titanium dioxide. Suitable flavorants include, but are not limited to, a natural flavor oil, an artificial flavor oil, a synthetic flavor oil, a flavoring aromatic, a flavoring oils, an oleoresin, plant extract, leaf extract, flower extract, fruit extract, citrus oil, spearmint oil, peppermint oil, Eucalyptus oil, nutmeg oil, allspice oil, mace, almond oil, menthol oil, citrus oil, lemon oil, orange oil, lime oil, grapefruit oil and/or combinations thereof. In embodiments, prenatal dosage forms may include citrus flavor, ginger flavor, berry flavor, cherry flavor, vanilla flavor or combinations thereof. Suitable sweeteners include, but are not limited to, agave syrup, Stevia, erythritol, xylitol, sorbitol, yacon syrup, aspartame, saccharin, cyclamate, sucralose, monk fruit extract and/or combinations thereof. Suitable preservatives include, but are not limited to, methylparaben, propylparaben, sodium methylhydroxybenzoate, sodium ethylhydroxybenzoate, sodium butyhydroxybenzoate, a quaternary ammonium compound, benzalkonium chloride and/or combinations thereof. In embodiments, suitable embrittlement agents include, but are not limited to, sorbitol, sorbitans, polyhydric alcohols and/or combinations thereof. Suitable disintegrants include, but are not limited to, polyvinylpyrrolidone, croscarmellose sodium, sodium starch glycolate and/or combinations thereof.

Prenatal dosage forms according to embodiments herein, can be in any suitable form known to those of ordinary skill in the art. In embodiments, the dosage forms may be in the form of liquids, creams, gels, solid form, tablets, chewable tablets, chewable gummies, film coated tablets, caplets, capsules, hard capsules, soft gel capsules, microcapsules, particles (e.g., microparticles, granules, powders, pellets, beads, spheres, microspheres, etc.), buccal tablets, troches, lozenges, suppositories, transdermal patches and combinations thereof. In certain embodiments, prenatal dosage forms as described herein may be in the form of a chewable tablet or a gummy having a citrus flavorant. In embodiments, the citrus flavorant may also be suitable as a taste-masking element to mask any metallic tastes from the vitamins in the prenatal dosage forms. According to embodiments, prenatal dosage forms can further include at least one of vitamin K2, at least one omega-3 fatty acid, potassium, red raspberry, Fubius idaeus, lutein, zeaxanthin, date fruit or combinations thereof. In embodiments, prenatal dosage forms further may be free of manganese and/or folate.

Prenatal dosage forms as described herein can be administered using any suitable route known to those of ordinary skill in the art. In embodiments, prenatal dosage forms can be administered orally, parenterally, intravenously, intradermally, subcutaneously, transdermally, rectally, intranasally, buccally, vaginally, by implanted reservoir and combinations thereof.

Some compounds compete for absorption following administration to a patient. For example, manganese and magnesium compete for absorption within the small intestine. Similarly, iron and calcium also compete for absorption. According to various embodiments (e.g., as further described below in Example 2), compounds useful for prenatal dosage forms as described herein may be combined (or separated) according to their individual absorption characteristics.

Most water-soluble vitamins are available for intestinal absorption from two sources: 1) the diet, and 2) synthesis by microbes in the large intestine or, in the case of ruminants, the rumen. These dual-origin vitamins include biotin, folic acid, pantothenic acid, riboflavin and thiamin. Ascorbic acid can be synthesized by many animals, but not by primates or guinea pigs, in which it is a true vitamin and must be obtained from dietary sources. Niacin is also a bit different—it can be synthesized within the body from tryptophan, but is also absorbed in the intestine from dietary sources. Water-soluble vitamins of dietary origin are absorbed predominantly in the small intestine, whereas those synthesized by microbes in the large intestine are absorbed there. For most of these vitamins, specific carrier-mediated transport systems have been identified that allow uptake from the intestinal lumen into the enterocyte and for export from the basolateral surface of the enterocyte. Some of these transporters are sodium-dependent, while others are not.

Fat-soluble compounds such as vitamins A, D, E and K are absorbed from the intestinal lumen using the same mechanisms used for absorption of other lipids. In short, they are incorporated into mixed micelles with other lipids and bile acids in the lumen of the small intestine and enter the enterocyte largely by diffusion. Within the enterocytge, they are incorporated into chylomicrons and exported via exocytosis into lymph.

The vast bulk of mineral absorption occurs in the small intestine. Mineral absorption is normally proportional to dietary intake, with two important distinctions—the absorption of iron and calcium, both of which can be regulated according to the needs of the body. Calcium absorption is related to the amount of specific binding protein within the enterocyte. The concentration of the calcium binding protein, which regulates calcium uptake from the gut, is secondary to vitamin D levels.

Iron supplementation can decrease the absorption of zinc and influence other antioxidant levels such as vitamin C. Iron absorption occurs in the duodenum and proximal jejunum. Following digestion, iron is in two forms. The first is heme iron bound to hemoglobin and myoglobin. The second form is free ionized iron in the ferrous and ferric state. Heme iron is absorbed by binding to a probable heme receptor, whereas free iron is likely to be absorbed by a specific carrier protein. Free iron is cytotoxic, so it is bound inside enterocytes to the large storage protein, apoferritin, or bound to transferrin for export to the bloodstream.

Copper (Cu) is an essential trace element for humans and animals. In the body, copper shifts between the cuprous (Cu′)+ and cupric (Cu′) forms, though the majority of the body's copper is in the Cu′ form. The ability of copper to easily accept and donate electrons explains its important role in oxidation-reduction (redox) reactions and in scavenging free radicals. In oxidation-reduction reactions involving copper-containing oxidases, copper is an essential cofactor. Copper enzymes regulate various physiologic pathways, such as energy production, iron metabolism, connective tissue maturation, and neurotransmission.

Copper deficiency can result from malnutrition, malabsorption, or excessive zinc intake and can be acquired or inherited. Symptoms include deficiencies in blood cells, bone and connective tissue abnormalities, and neurologic disorders. Marginal copper imbalance has been linked to impaired immune function, bone demineralization, and increased risk of cardiovascular and neurodegenerative diseases.

Four copper-containing enzymes, known as multi-copper oxidases (MCO) or ferroxidases, have the capacity to oxidize ferrous iron (Fe²⁺) to ferric iron (Fe³⁺), the form of iron that can be loaded onto the protein transferrin for transport to the site of red blood cell formation. The MCO family includes the circulating ceruloplasmin (which represents ˜90% of plasma copper), the membrane-bound ceruloplasmin (called GPI-ceruloplasmin), and two proteins called Hephaestin and Zyklopen found in the intestine and the placenta, respectively.

Although vitamin C supplements have produced copper deficiency in guinea pigs, the effect of vitamin C supplements on copper nutritional status in humans is less clear. Two small studies indicate that the oxidase activity of ceruloplasmin may be impaired by relatively high doses of supplemental vitamin C.

In some embodiments, water-soluble compounds may be formulated into at least one first dosage form. According to some embodiments, the at least one first dosage form may be formulated so as not to include one or more of fat soluble compounds, iodine, minerals, iron, vitamin C and/or copper. The at least one first dosage form may include one or more of vitamins B1, B2, B3, B5, B6, B12, folate, folic acid, biotin, myo-inositol and/or D-chiro-inositol. In some embodiments, the at least one first dosage form may include vitamin B1 in an amount of about 1 mg to about 10 mg, vitamin B2 in an amount of about 1 mg to about 10 mg, vitamin B3 in an amount of about 15 mg to about 35 mg, vitamin B6 in an amount of about 15 mg to about 100 mg, folate in an amount of about 400 mcg to about 1000 mcg, folic acid in an amount of about 200 mcg to about 500 mcg, vitamin B12 in an amount of about 50 mcg to about 300 mcg, biotin in an amount of about 100 mcg to about 600 mcg, myo-inositol in an amount of about 1000 mg to about 4 g and/or D-Chiro-Inositol in an amount of about 25 mg to about 4 g.

In some embodiments, certain minerals may be formulated into at least one second dosage form. According to some embodiments, the at least one second dosage form may be formulated so as not to include one or more of certain water-soluble compounds, fat-soluble compounds, iodine and minerals other than iron and copper. The at least one second dosage form may include one or more of vitamin C, iron and/or copper. In some embodiments, the at least one second dosage form may include vitamin C in an amount of about 50 mg to about 2000 mg, iron in an amount of about 10 mg to about 45 mg and/or copper in an amount of about 0.5 mg to about 10 mg. In some embodiments, the first and second dosage forms may be suitable for consecutive or co-administration in the morning. The term “morning” as used herein with reference to administration refers to about 3 AM to about 11:00 AM. The term “afternoon” as used herein with reference to administration refers to about 11:00 AM to about 4:00 PM. The term “evening” as used herein with reference to administration refers to about 4:00 PM to about 12:00 AM.

In some embodiments, minerals other than iron and copper may be formulated into at least one third dosage form. According to some embodiments, the at least one third dosage form may be formulated so as not to include one or more of water-soluble compounds, fat-soluble compounds, iodine, iron and copper. The at least one third dosage form may include one or more of choline, calcium, magnesium, zinc, selenium, chromium and/or potassium. In some embodiments, the at least one third dosage form may include about 350 mg to about 600 mg choline, about 300 mg to about 1000 mg calcium, about 250 mg to about 450 mg magnesium, about 100 mcg to about 300 mcg zinc, about 100 mcg to about 300 mcg selenium, about 20 mcg to about 120 mcg chromium and/or about 0 mg to about 60 mg potassium.

In some embodiments, iodine may be formulated into at least one fourth dosage form. The iodine may be formulated by itself into a separate dosage form to enable those with thyroid issues to leave out the iodine compound. Medical advisors, in particular, the CDC and NIH, are torn about the need to include iodine within a prenatal dosage form. Some recent insight indicates that many people in the United States actually have fibroid issues caused by too much iodine in the diet. Furthermore, in some people who have an underactive thyroid, too much iodine can cause or worsen their condition. For example, the patient's healthcare provider may prescribe a prenatal vitamin that is free of iodine. The patient can simply toss and/or not administer the at least one fourth dosage form that contains iodine and is free of water-soluble compounds, fat-soluble compounds and minerals. In one or more embodiments, the at least one fourth dosage form may contain about 50 mcg to about 250 mcg iodine.

In some embodiments, the fat-soluble compounds may be formulated into at least one fifth dosage form. According to some embodiments, the at least one fifth dosage form may be free of one or more of water-soluble compounds, iodine and/or minerals. The at least one fifth dosage form may include one or more of vitamins A, D, E, K1, K2, omega-3 eicosapentaenoic acid, omega-3 docosahexaenoic acid, coenzyme Q10 (CoQ10), lutein and/or zeaxanthin. In some embodiments, the at least one fifth dosage form may include about 500 mcg to about 1000 mcg vitamin A (beta carotene), about 200 mcg to about 600 mcg vitamin A (retinyl plamitate), about 50 mcg to about 500 mcg vitamin D, about 50 mg to about 500 mg vitamin E, about 30 mcg to about 90 mcg vitamin K1, about 30 mcg to about 90 mcg vitamin K2, about 150 mg to about 750 mg eicosapentaenoic acid, about 150 mg to about 750 mg docosahexaenoic acid, about 100 mg to about 500 mg CoQ10, about 2 mg to about 10 mg lutein and/or about 1 mg to about 4 mg zeaxanthin. In some embodiments, the third, fourth and fifth dosage forms may be suitable for consecutive or co-administration in the evening.

In some embodiments, the first to fifth dosage forms may be contained in three packets. The first packet (e.g., step one) may be formulated for administration in the morning and may contain the first and/or second dosage forms. The second packet (e.g., step two) may contain the second and/or third dosage form and may be formulated for administration about 30 minutes to about two hours before or after a meal. The third packet (e.g., step three) may include one or more of the third to fifth dosage forms and may be formulated for administration together with a meal, for example, with lunch, an afternoon snack or dinner. Alternatively, as described herein, the first to fifth dosage forms may be divided between two packets the first formulated for morning administration and the second formulated for afternoon or evening administration.

Grouping the water soluble components together in the first to fifth dosage forms as described herein can improve adsorption of the compounds within the formulations. Separating manganese and magnesium as well as iron and calcium can ensure that a mother taking the dosage forms at one or more of the stages of pregnancy/reproduction can absorb the appropriate amounts of these compounds. Dosage forms having compounds grouped as set forth in the first to fifth dosage forms and administered separately (e.g., morning, afternoon and evening), can provide improved absorption over conventional prenatal supplements. In some embodiments, prenatal vitamin kits are do not contain iodine and the one or more fourth dosage form.

### Methods of Preparing Prenatal Dosage Forms

Prenatal dosage forms according to embodiments herein may be prepared using any suitable methods or combination of methods known to those of ordinary skill in the art. Any of the components of the prenatal dosage forms described herein can be used as supplied commercially, or can be preprocessed by agglomeration, air suspension chilling, air suspension drying, balling, coacervation, comminution, compression, pelletization, cryopelletization, extrusion, wet granulation, dry granulation, homogenization, inclusion complexation, lyophilization, melting, mixing, molding, pan coating, solvent dehydration, sonication, spheronization, spray chilling, spray congealing, spray drying, or other processes known in the art. In embodiments, the components, for example, in particle form, may be compressed into tablets or caplets. In yet further embodiments, the components may be encapsulated using any suitable method known to those or ordinary skill in the art.

Methods of Supplementing Nutrition with Prenatal Dosage Forms

According to embodiments, disclosed herein are prenatal nutritional supplements and programs that can be personalized by month and can include a mobile/digital platform to optimize health outcomes for women at every stage of the pregnancy cycle from pre-conception/fertility to post-pregnancy/breastfeeding. Methods of supplementing the nutrition of a prenatal, pregnant or lactating woman can include administering a first prenatal dosage form to the woman for about 1 week to about 12 months; administering a second prenatal dosage form to the woman for about 1 week to about 16 weeks; and administering a third prenatal dosage form to the woman for about 1 week to about 9 months, wherein the first formulation, the second formulation and third formulation are administered over consecutive time periods.

According to embodiments, the first prenatal dosage form may be formulated for one or more of the pre-pregnancy stage (about 3-12 months prior to conception to increase fertility), the first week of pregnancy, the first trimester (e.g., the first 13-14 weeks of pregnancy), or the first four months of pregnancy (e.g., the first 16 weeks). In embodiments, the first prenatal dosage form may be formulated for the first 4-5 weeks of pregnancy. In further embodiments, the first prenatal dosage form may be formulated for pre-conception to be taken for a period of 3-12 months to increase fertility and while trying to conceive. The term “first prenatal dosage form” as used herein can mean a single dosage form or a plurality of dosage forms. The plurality of dosage forms may all contain the same compounds in the same amounts, may each contain different compounds, may each contain a different combination of compounds, or a combination of the foregoing (e.g., two dosage forms may be the same while the rest are all different).

According to embodiments, the second prenatal dosage form may be formulated for one or more of the first week of pregnancy, the second week of pregnancy, the second two weeks of pregnancy, the first trimester (e.g., the first 13-14 weeks of pregnancy) or the second trimester (e.g., weeks 14-27 of pregnancy). In embodiments, the second prenatal dosage form may be formulated for the second 4-5 weeks of pregnancy (i.e., weeks 8-9) administered consecutively with the first prenatal dosage form. In further embodiments, the second prenatal dosage form may be formulated for the second trimester of pregnancy (i.e., weeks 14-27) and administered consecutively with the first prenatal dosage form when formulated for the first trimester of pregnancy (e.g., weeks 1-14). The term “second prenatal dosage form” as used herein can mean a single dosage form or a plurality of dosage forms. The plurality of dosage forms may all contain the same compounds in the same amounts, may each contain different compounds, may each contain a different combination of compounds, or a combination of the foregoing (e.g., two dosage forms may be the same while the rest are all different).

According to embodiments, the third prenatal dosage form may be formulated for one or more of the second week of pregnancy, the third week of pregnancy, the fifth and sixth weeks of pregnancy, the second trimester (e.g., the weeks 14-27 of pregnancy), the third trimester (e.g., weeks 28-40 of pregnancy) and/or during the breastfeeding stage for about 3-12 months following birth. In embodiments, the third prenatal dosage form may be formulated for the third 4-5 weeks of pregnancy (i.e., weeks 9-14) administered consecutively with the second prenatal dosage form. In further embodiments, the third prenatal dosage form may be formulated for the third trimester of pregnancy (i.e., weeks 27-40) and administered consecutively with the second prenatal dosage form when formulated for the second trimester of pregnancy (e.g., weeks 14-27). The term “third prenatal dosage form” as used herein can mean a single dosage form or a plurality of dosage forms. The plurality of dosage forms may all contain the same compounds in the same amounts, may each contain different compounds, may each contain a different combination of compounds, or a combination of the foregoing (e.g., two dosage forms may be the same while the rest are all different).

In embodiments, the methods of supplementing the nutrition of a prenatal, pregnant or lactating woman can include administering a fourth prenatal dosage form to the woman for about 1 week to about 10 weeks. According to embodiments, the fourth prenatal dosage form may be formulated for one or more of the third week of pregnancy, the fourth week of pregnancy, the seventh and eighth weeks of pregnancy, weeks 31-40 of pregnancy and/or during the breastfeeding stage for about 3-12 months following birth. In embodiments, the fourth prenatal dosage form may be formulated for the fourth 4-5 weeks of pregnancy (i.e., weeks 13-20) administered consecutively with the third prenatal dosage form. The term “fourth prenatal dosage form” as used herein can mean a single dosage form or a plurality of dosage forms. The plurality of dosage forms may all contain the same compounds in the same amounts, may each contain different compounds, may each contain a different combination of compounds, or a combination of the foregoing (e.g., two dosage forms may be the same while the rest are all different).

In embodiments, the methods of supplementing the nutrition of a prenatal, pregnant or lactating woman can include administering a fifth prenatal dosage form to the woman for about 1 week to about 8 weeks. According to embodiments, the fifth prenatal dosage form may be formulated for one or more of the fourth week of pregnancy, the fifth week of pregnancy, the ninth and tenth weeks of pregnancy, weeks 32-40 of pregnancy and/or during the breastfeeding stage for about 3-12 months following birth. In embodiments, the fifth prenatal dosage form may be formulated for the fifth 4-5 weeks of pregnancy (i.e., weeks 21-25) administered consecutively with the fourth prenatal dosage form. The term “fifth prenatal dosage form” as used herein can mean a single dosage form or a plurality of dosage forms. The plurality of dosage forms may all contain the same compounds in the same amounts, may each contain different compounds, may each contain a different combination of compounds, or a combination of the foregoing (e.g., two dosage forms may be the same while the rest are all different).

According to embodiments, the methods of supplementing the nutrition of a prenatal, pregnant or lactating woman can include administering a sixth prenatal dosage form to the woman for about 1 week to about 7 weeks. In embodiments, the sixth prenatal dosage form may be formulated for one or more of the fifth week of pregnancy, the sixth week of pregnancy, the eleventh and twelfth weeks of pregnancy, weeks 33-40 of pregnancy and/or during the breastfeeding stage for about 3-12 months following birth. In embodiments, the sixth prenatal dosage form may be formulated for the sixth 4-5 weeks of pregnancy (i.e., weeks 25-29) administered consecutively with the fifth prenatal dosage form. The term “sixth prenatal dosage form” as used herein can mean a single dosage form or a plurality of dosage forms. The plurality of dosage forms may all contain the same compounds in the same amounts, may each contain different compounds, may each contain a different combination of compounds, or a combination of the foregoing (e.g., two dosage forms may be the same while the rest are all different).

In embodiments, the methods of supplementing the nutrition of a prenatal, pregnant or lactating woman can include administering a seventh prenatal dosage form to the woman for about 1 week to about 6 weeks. According to embodiments, the seventh prenatal dosage form may be formulated for one or more of the sixth week of pregnancy, the seventh week of pregnancy, the thirteenth and fourteenth weeks of pregnancy, weeks 34-40 of pregnancy and/or during the breastfeeding stage for about 3-12 months following birth. In embodiments, the seventh prenatal dosage form may be formulated for the seventh 4-5 weeks of pregnancy (i.e., weeks 28-35) administered consecutively with the sixth prenatal dosage form. The term “seventh prenatal dosage form” as used herein can mean a single dosage form or a plurality of dosage forms. The plurality of dosage forms may all contain the same compounds in the same amounts, may each contain different compounds, may each contain a different combination of compounds, or a combination of the foregoing (e.g., two dosage forms may be the same while the rest are all different).

According to embodiments, the methods of supplementing the nutrition of a prenatal, pregnant or lactating woman can include administering an eighth prenatal dosage form to the woman for about 1 week to about 5 weeks. In embodiments, the eighth prenatal dosage form may be formulated for one or more of the seventh week of pregnancy, the eighth week of pregnancy, the fifteenth and sixteenth weeks of pregnancy, weeks 35-40 of pregnancy and/or during the breastfeeding stage for about 3-12 months following birth. In embodiments, the eighth prenatal dosage form may be formulated for the eighth 4-5 weeks of pregnancy (i.e., weeks 32-40) administered consecutively with the seventh prenatal dosage form. The term “eighth prenatal dosage form” as used herein can mean a single dosage form or a plurality of dosage forms. The plurality of dosage forms may all contain the same compounds in the same amounts, may each contain different compounds, may each contain a different combination of compounds, or a combination of the foregoing (e.g., two dosage forms may be the same while the rest are all different).

In embodiments, the methods of supplementing the nutrition of a prenatal, pregnant or lactating woman can include administering a ninth prenatal dosage form to the woman for about 1 week to about 5 weeks. According to embodiments, the ninth prenatal dosage form may be formulated for one or more of the eighth week of pregnancy, the ninth week of pregnancy, the seventeenth and eighteenth weeks of pregnancy, weeks 36-40 of pregnancy and/or during the breastfeeding stage for about 3-12 months following birth. In embodiments, the ninth prenatal dosage form may be formulated for the ninth 4-5 weeks of pregnancy (i.e., weeks 36-40) administered consecutively with the eighth prenatal dosage form. The term “ninth prenatal dosage form” as used herein can mean a single dosage form or a plurality of dosage forms. The plurality of dosage forms may all contain the same compounds in the same amounts, may each contain different compounds, may each contain a different combination of compounds, or a combination of the foregoing (e.g., two dosage forms may be the same while the rest are all different).

According to embodiments, the methods of supplementing the nutrition of a prenatal, pregnant or lactating woman can include administering each of the first to ninth prenatal dosage forms to the woman for about 4 weeks to about 5 weeks each. Each of the first to ninth prenatal dosage forms can administered over consecutive time periods. For example, the first prenatal dosage form can be administered to a pregnant woman for weeks 1 to 5 of pregnancy, the second prenatal dosage from can be administered to the woman for weeks 5 to 9, the third prenatal dosage form can be administered to the woman for weeks 8 to 14, the fourth prenatal dosage form can be administered to the woman for weeks 13 to 18, the fifth prenatal dosage form can be administered to the woman for weeks 17 to 23, the sixth prenatal dosage form can be administered to the woman for weeks 22 to 27, the seventh prenatal dosage form can be administered to the woman for weeks 26 to 32, the eighth prenatal dosage form can be administered to the woman for weeks 31 to 36 and the ninth prenatal dosage form can be administered to the woman for weeks 35 to 40.

There is some evidence showing that supplementing with folate has a cumulative effect and continuing folate into the third trimester can increase gestational weight and can lead to allergies in the baby. According to embodiments, prenatal dosage forms as described herein, when formulated for the third trimester of pregnancy, are free of folate and folic acid. For example, the third prenatal dosage form may be formulated for the third trimester of pregnancy and may be free of folate and folic acid. In embodiments, the fourth, fifth, sixth, seventh, eighth and ninth prenatal dosage forms, when formulated for the third trimester, are free of folate and folic acid.

According to embodiments, the methods of supplementing the nutrition of a prenatal, pregnant or lactating woman can further include administering each of a tenth to a twentieth prenatal dosage form to a pregnant woman for about 2 weeks to about 4 weeks each. The tenth to twentieth prenatal dosage forms can be administered consecutively following the administration of the twentieth dosage form as discussed above. For example, 20 prenatal dosage forms can be administered consecutively over a period of 400 weeks, such that each prenatal dosage form is administered for two weeks.

In yet further embodiments, the methods of supplementing the nutrition of a prenatal, pregnant or lactating woman can further include administering each of a twenty-first to a fortieth prenatal dosage form to a pregnant woman for about 1 week to 2 weeks each. In embodiments, each of the first to fortieth prenatal dosage forms are administered over consecutive time periods. The twenty-first to fortieth prenatal dosage forms can be administered consecutively following the administration of the twentieth dosage form as discussed above. For example, 40 prenatal dosage forms can be administered consecutively over a period of 40 weeks, such that each prenatal dosage form is administered for one week. The first to fortieth prenatal dosage forms each can be a single dosage form or a plurality of dosage forms.

According to some embodiments, methods of supplementing the nutrition of a prenatal, pregnant or lactating woman may include providing a kit containing about 1 to about 90 containers (e.g., packets, bottles, blister packs, pouches, etc.) of dosage forms for one or more stages of pregnancy (i.e., pre-pregnancy, Trimester 1, Trimester 2, Trimester 3 and post-pregnancy). The kit can include any combination of the first to fifth dosage forms as described herein for either twice daily or three times daily administration. Included in the kit may be instructions advising a patient to administer the contents of certain containers at certain times and with or without food. The proper administration of the dosage forms within the kits may improve absorption of the compounds contained therein as compared to conventional prenatal vitamins.

Kits for Supplementing Nutrition with Prenatal Dosage Forms

Further disclosed herein are kits for supplementing nutrition with prenatal dosage forms as described herein. The kits can include any of the prenatal dosage forms according to embodiments herein. According to embodiments, kits can include a first prenatal dosage form formulated for daily administration for a period of about 1 week to about 12 months, a second prenatal dosage form formulated for daily administration for about 1 week to about 16 weeks, and a third prenatal dosage form formulated for daily administration for about 1 week to about 9 months.

In embodiments, kits as described herein can further include a fourth prenatal dosage form formulated for daily administration for about 1 week to about 10 weeks, a fifth prenatal dosage form formulated for daily administration for about 1 week to about 8 weeks, a sixth prenatal dosage form formulated for daily administration for about 1 week to about 7 weeks, a seventh prenatal dosage form formulated for daily administration for about 1 week to about 6 weeks, an eighth prenatal dosage form formulated for daily administration for about 1 week to about 5 weeks and a ninth prenatal dosage form formulated for daily administration for about 1 week to about 5 weeks. According to embodiments, kits as described herein can include up to forty formulations as described herein.

In embodiments, the kits can further include instructions for administering the formulations included therein. For example, a kit may include 11 formulations. In embodiments, the instructions can direct a woman to administer the fertility support formulation for a period of three (3) to six (6) months while trying to conceive. The instructions further can direct the woman, once pregnant, to take each of the first to ninth formulations consecutively over nine months, for example, for a period of about 4 weeks to about 5 weeks each, during her pregnancy. The instructions additionally can direct the woman to administer the post-natal formulation for a period of 3 to 12 months following birth, while breastfeeding the baby.

According to some embodiments, further described herein are one or more kit containing about 1 to about 360, about 7 to about 180, or about 30 to about 90 containers (e.g., packets, bottles, blister packs, etc.), or any individual value or sub-range within these ranges, of dosage forms for one or more stages of pregnancy (i.e., pre-pregnancy, Trimester 1, Trimester 2, Trimester 3 and post-pregnancy). The one or more kits can include any combination of the first to fifth dosage forms as described herein for either twice daily or three times daily administration. Included in the kit may be instructions advising a patient to administer the contents of certain containers at certain times and with or without food. The proper administration of the dosage forms within the kits may improve absorption of the compounds contained therein as compared to conventional prenatal vitamins.

### EXAMPLES

### Example 1—Prenatal Dosage Forms and Kits (Prophetic)

Eleven (11) formulations of prenatal dosage forms are prepared. A fertility support formulation can be formulated to include the components in the amounts as shown in Table 1. Nine (9) of the 11 formulations each can be formulated for months 1 to 9 (e.g., 4-5 weeks each) of the pregnancy as shown in Table 1.1. A post-natal formulation can be formulated as shown in Table 1.2. The 11 formulations may be packaged together as a kit, which may include instructions to administer the fertility support formulation for a period of three (3) to six (6) months while trying to conceive. The instructions further direct the woman, once pregnant, to take each of the first to ninth formulations consecutively over nine months, for example, for a period of about 4 weeks to about 5 weeks each, during her pregnancy. The instructions additionally direct the woman to administer the post-natal formulation for a period of 3 to 12 months following birth, while breastfeeding the baby.

TABLE 1.1
Embodiments of Prenatal dosage forms

| | | | Month | | Month | | Month | | Month | | Month | | Month | | Month | | Month | | Month | |
|---|---|---|---|---|---|---|---|---|---|---|---|---|---|---|---|---|---|---|---|---|
| Component | Fertility Support | | 1 | | 2 | | 3 | | 4 | | 5 | | 6 | | 7 | | 8 | | 9 | |
| Vitamin A (e.g., as Beta Carotene from Organic | 800 | mcg | 1300 | mcg | 1300 | mcg | 1300 | mcg | 1400 | mcg | 1400 | mcg | 1400 | mcg | 1500 | mcg | 1500 | mcg | 1500 | mcg |
| Food Blend) | | | | | | | | | | | | | | | | | | | | |
| Vitamin C (e.g., from Organic Food Blend) | 50 | mg | 60 | mg | 70 | mg | 80 | mg | 90 | mg | 100 | mg | 110 | mg | 120 | mg | 120 | mg | 120 | mg |
| Vitamin D (e.g., D3 from Lichen, D2 from Organic | 50 | mcg | 50 | mcg | 50 | mcg | 50 | mcg | 50 | mcg | 50 | mcg | 50 | mcg | 50 | mcg | 50 | mcg | 50 | mcg |
| Agaricus bisporus) | | | | | | | | | | | | | | | | | | | | |
| Vitamin E (e.g., from Organic Food Blend) | 20 | mg | 10 | mg | 10 | mg | 10 | mg | 15 | mg | 15 | mg | 15 | mg | 20 | mg | 20 | mg | 20 | mg |
| Vitamin K1 | 10 | mcg | 45 | mcg | 45 | mcg | 45 | mcg | 45 | mcg | 45 | mcg | 45 | mcg | 60 | mcg | 70 | mcg | 75 | mcg |
| Vitamin K2 | 90 | mcg | 45 | mcg | 45 | mcg | 45 | mcg | 45 | mcg | 45 | mcg | 45 | mcg | 30 | mcg | 20 | mcg | 25 | mcg |
| Thiamin (e.g., Vitamin B2 from Organic Food Blend) | 2 | mg | 3 | mg | 4 | mg | 5 | mg | 3 | mg | 3 | mg | 3 | mg | 3 | mg | 3 | mg | 3 | mg |
| Riboflavin (e.g., Vitamin B2 from Organic Food Blend) | 2 | mg | 2 | mg | 2 | mg | 2 | mg | 2 | mg | 2 | mg | 2 | mg | 2 | mg | 2 | mg | 2 | mg |
| Niacin (e.g., from Organic Food Blend) | 18 | mg | 12 | mg | 12 | mg | 12 | mg | 15 | mg | 15 | mg | 15 | mg | 18 | mg | 18 | mg | 18 | mg |
| Vitamin B6 (e.g., from Organic Food Blend) | 10 | mg | 20 | mg | 15 | mg | 10 | mg | 9 | mg | 8 | mg | 7 | mg | 6 | mg | 5 | mg | 4 | mg |
| Folate (e.g., from Organic Food Blend) | 800 | mcg | 800 | mcg | 800 | mcg | 800 | mcg | 700 | mcg | 700 | mcg | 700 | mcg | 600 | mcg | 600 | mcg | 600 | mcg |
| Vitamin B12 (e.g., methylcobal amin from | 50 | mcg | 45 | mcg | 40 | mcg | 35 | mcg | 30 | mcg | 25 | mcg | 20 | mcg | 15 | mcg | 10 | mcg | 5 | mcg |
| Saccharomyces cerevisae) | | | | | | | | | | | | | | | | | | | | |
| Biotin (e.g, from Organic Food Blend) | 35 | mcg | 30 | mcg | 30 | mcg | 30 | mcg | 30 | mcg | 30 | mcg | 30 | mcg | 30 | mcg | 30 | mcg | 30 | mcg |
| Pantothenic Acid (e.g, from Organic Food Blend) | 10 | mg | 10 | mg | 10 | mg | 10 | mg | 12.5 | mg | 12.5 | mg | 12.5 | mg | 15 | mg | 15 | mg | 15 | mg |
| Choline (e.g., as Choline Bitartrate) | 300 | mg | 300 | mg | 300 | mg | 300 | mg | 300 | mg | 300 | mg | 300 | mg | 300 | mg | 300 | mg | 300 | mg |
| Calcium (e.g., as Calcium Carbonate) | 500 | mg | 500 | mg | 500 | mg | 500 | mg | 500 | mg | 500 | mg | 500 | mg | 500 | mg | 500 | mg | 500 | mg |
| Iron (e.g., from Organic Food Blend) | 30 | mg | 30 | mg | 30 | mg | 30 | mg | 30 | mg | 30 | mg | 30 | mg | 30 | mg | 30 | mg | 30 | mg |
| Iodine (e.g., from Organic Food Blend) | 150 | mcg | 160 | mcg | 170 | mcg | 180 | mcg | 190 | mcg | 200 | mcg | 210 | mcg | 220 | mcg | 220 | mcg | 220 | mcg |
| Magnesium (e.g., as Magnesium Aspartate) | 320 | mg | 320 | mg | 320 | mg | 320 | mg | 350 | mg | 350 | mg | 350 | mg | 370 | mg | 370 | mg | 370 | mg |
| Zinc (e.g., from Organic Food Blend) | 10 | mg | 10 | mg | 10 | mg | 10 | mg | 20 | mg | 20 | mg | 20 | mg | 30 | mg | 30 | mg | 30 | mg |
| Selenium (e.g., from Organic Food Blend) | 100 | mcg | 100 | mcg | 100 | mcg | 100 | mcg | 100 | mcg | 100 | mcg | 100 | mcg | 100 | mcg | 100 | mcg | 100 | mcg |
| Chromium (e.g., from Organic Food Blend) | — | | — | | — | | — | | — | | 20 | mcg | 20 | mcg | 30 | mcg | 30 | mcg | 40 | mcg |
| Copper (e.g., as copper gluconate, USP) | 1 | mg | 1 | mg | 1 | mg | 1 | mg | 1 | mg | 1 | mg | 1 | mg | 1 | mg | 1 | mg | 1 | mg |
| >ALA | 100 | mg | 100 | mg | 100 | mg | 100 | mg | 100 | mg | 100 | mg | 100 | mg | 100 | mg | 100 | mg | 100 | mg |
| >EPA | 100 | mg | 150 | mg | 150 | mg | 150 | mg | 150 | mg | 150 | mg | 150 | mg | 150 | mg | 150 | mg | 150 | mg |
| >DHA | 100 | mg | 200 | mg | 200 | mg | 200 | mg | 200 | mg | 200 | mg | 200 | mg | 250 | mg | 250 | mg | 250 | mg |
| Potassium | 99 | mg | 99 | mg | 99 | mg | 99 | mg | 99 | mg | 99 | mg | 99 | mg | 99 | mg | 99 | mg | 99 | mg |
| Red Raspberry (e.g., Rubusidaeus, leaf) | — | | — | | — | | — | | — | | — | | — | | 100 | mg | 250 | mg | 250 | mg |
| Lutein (e.g., from marigold extract, tagetes | 5 | mg | 6 | mg | 7 | mg | 8 | mg | 8 | mg | 8 | mg | 8 | mg | 8 | mg | 8 | mg | 8 | mg |
| erecta, flower) | | | | | | | | | | | | | | | | | | | | |
| Zeaxanthin (e.g., from marigold extract, tagetes | 2 | mg | 2 | mg | 2 | mg | 2 | mg | 2 | mg | 2 | mg | 2 | mg | 2 | mg | 2 | mg | 2 | mg |
| erecta, flower) | | | | | | | | | | | | | | | | | | | | |

TABLE 1-2
Post-Natal Breastfeeding Formulation

| | Post-Natal | |
|---|---|---|
| Component | (Breastfeeding) | |
| Vitamin A (e.g., as Beta | 1500 | mcg |
| Carotene from Organic | | |
| Food Blend) | | |
| Vitamin C (e.g., from | 120 | mg |
| Organic Food Blend) | | |
| Vitamin D (e.g., D3 from | 50 | mcg |
| Lichen, D2 from Organic | | |
| Agaricus bisporus) | | |
| Vitamin E (e.g., from | 20 | mg |
| Organic Food Blend) | | |
| Vitamin K1 | 75 | mcg |
| Vitamin K2 | 25 | mcg |
| Thiamin (e.g., Vitamin B2 | 2 | mg |
| from Organic Food | | |
| Blend) | | |
| Riboflavin (Vitamin B2 | 3 | mg |
| from Organic Food | | |
| Blend) | | |
| Niacin (e.g., from Organic | 30 | mg |
| Food Blend) | | |
| Vitamin B6 (e.g., from | 2 | mg |
| Organic Food Blend) | | |
| Folate (e.g., from Organic | 500 | mcg |
| Food Blend) | | |
| Vitamin B12 (e.g., as | 5 | mcg |
| Methylcobalamin from | | |
| Saccharomyces cerevisae) | | |
| Biotin (e.g., from Organic | 50 | mcg |
| Food Blend) | | |
| Pantothenic Acid (e.g., | 15 | mg |
| from Organic Food | | |
| Blend) | | |
| Choline (e.g., as Choline | 400 | mg |
| Bitartrate) | | |
| Calcium (e.g., as Calcium | 500 | mg |
| Carbonate) | | |
| Iron (e.g., from Organic | 30 | mg |
| Food Blend) | | |
| Iodine (e.g., from Organic | 290 | mcg |
| Food Blend) | | |
| Magnesium (e.g, as | 390 | mg |
| Magnesium Aspartate) | | |
| Zinc (e.g., from Organic | 40 | mg |
| Food Blend) | | |
| Selenium (e.g., from | 100 | mcg |
| Organic Food Blend) | | |
| Chromium (e.g., from | 40 | mcg |
| Organic Food Blend) | | |
| Copper (e.g., as copper | 1 | mg |
| gluconate, USP) | | |
| >ALA | 100 | mg |
| >EPA | 150 | mg |
| >DHA | 250 | mg |
| Potassium | 99 | mg |
| Red Raspberry (e.g., | 300 | mg |
| Rubus idaeus, leaf) | | |
| Lutein (e.g., from | 8 | mg |
| marigold extract, tagetes | | |
| erecta, flower) | | |
| Zeaxanthin (e.g., from | 2 | mg |
| marigold extract, tagetes | | |
| erecta, flower) | | |

### Example 2—Two Steps Daily Prenatal Dosage Forms and Kits

Several kits were prepared, each kit containing dosage forms formulated for a particular stage of reproduction: 1) pre-conception; 2) pregnancy Trimester 1; 3) pregnancy Trimester 2; 4) pregnancy Trimester 3; and 5) post-natal. The kits included custom-formulated phased prenatal vitamins with a time of day protocol to optimize absorption. The kits included two steps (e.g., vitamin packs, collection of dosage forms containing vitamins, etc.) for daily administration based on the time of day—morning (AM) and afternoon/evening (PM). The compositions of the AM steps for each kit is set forth in Table 2.1 and for the PM steps for each kit is set forth in Table 2.2.

The pre-conception kit included dosage forms having coenzyme Q10 (CoQ10) and Myo- and D-Chiro inositol as well as increased levels (i.e., in about the amounts as set forth in Tables 2.1 and 2.2) of Vitamin D, Vitamin E and Chromium. The Trimester 1 kit included dosage forms having increased choline, B1, B2, B6, biotin and B5 as compared to the pre-conception kit and decreased Vitamin D, Vitamin E and Chromium also as compared to the pre-conception kit. The Trimester 2 kit included dosage forms with increased choline, calcium, zinc, chromium, B1, B2, B12 and biotin as compared to the Trimester 1 kit. The Trimester 2 kit also included decreased B6 for maintenance as compared to the Trimester 1 kit. The Trimester 3 kit contained dosage forms having increased levels of Vitamin A, Vitamin D, magnesium, lutein, zeaxanthin, Vitamin K1 and K2, choline, calcium, zinc, chromium and potassium as compared to the Trimester 2 kit. The post-natal kit contained dosage forms including increased level of iodine, Vitamin A, Vitamin D, Vitamin E, Vitamin K1 and K2, B1, B2, B12, biotin (e.g., for hair loss) and B5 as compared to the Trimester 3 kit.

TABLE 2.1
Components in the dosage forms from the AM step

| | | | | | | |
|---|---|---|---|---|---|---|
| Ingredient | Form | Preconception | Trimester 1 | Trimester 2 | Trimester 3 | Post-Natal |

Water Soluble/B Vitamins

| | | | | | | | | | | | |
|---|---|---|---|---|---|---|---|---|---|---|---|
| Vitamin B1 (Thiamin) | Thiamin HCl | 4 | mg | 5 | mg | 6 | mg | 6 | mg | 8 | mg |
| Vitamin B2 (Riboflavin) | Riboflavin-5-phosphate | 5 | mg | 6 | mg | 7 | mg | 7 | mg | 8 | mg |
| Vitamin B3 (Niacin) | Niacinamide | 32 | mg | 32 | mg | 30 | mg | 30 | mg | 30 | mg |
| Vitamin B6 | Pyridoxal-5-phosphate | 30 | mg | 40 | mg | 30 | mg | 30 | mg | 30 | mg |
| Folate | L-5- | 600 | mcg | 600 | mcg | 600 | mcg | 600 | mcg | 600 | mcg |
| | methyltetra hydrofolate | | | | | | | | | | |
| Folic Acid | synthetic | 400 | mcg | 400 | mcg | 400 | mcg | 400 | mcg | 400 | mcg |
| Vitamin B12 | Methylcobalamin + | 150 | mcg | 150 | mcg | 175 | mcg | 175 | mcg | 200 | mcg |
| | Adenosylcobalamin | | | | | | | | | | |
| Biotin | Biotin | 200 | mcg | 225 | mcg | 250 | mcg | 250 | mcg | 500 | mcg |
| B5 (Pantothenic Acid) | D-Calcium Pantothenate | 100 | mg | 125 | mg | 125 | mg | 125 | mg | 150 | mg |

| | | | | | | | |
|---|---|---|---|---|---|---|---|
| Myo-Inositol | Myo-Inositol | 2000 | mg | — | — | — | — |
| D-Chiro-lnositol | D-Chiro-lnositol | 50 | mg | — | — | — | — |

Minerals

| | | | | | | | | | | | |
|---|---|---|---|---|---|---|---|---|---|---|---|
| Vitamin C | Ascorbic Acid | 120 | mg | 120 | mg | 120 | mg | 120 | mg | 120 | mg |
| Iron | Ferrous bisglycinate | 30 | mg | 30 | mg | 30 | mg | 30 | mg | 30 | mg |
| | (e.g., chelate form | | | | | | | | | | |
| | with Ferrochel) | | | | | | | | | | |
| Copper | Copper bisglycinate | 1 | mg | 1 | mg | 1 | mg | 1 | mg | 1 | mg |
| | chelate | | | | | | | | | | |

TABLE 2.2
Components in the dosage forms from the PM step

| | | | | | | |
|---|---|---|---|---|---|---|
| Ingredient | Form | Preconception | Trimester 1 | Trimester 2 | Trimester 3 | Post-Natal |

Minerals

| | | | | | | | | | | | |
|---|---|---|---|---|---|---|---|---|---|---|---|
| Choline | CDP Choline | 450 | mg | 500 | mg | 525 | mg | 550 | mg | 550 | mg |
| Calcium | Calcium Citrate | 500 | mg | 500 | mg | 600 | mg | 750 | mg | 750 | mg |
| Magnesium | Magnesium Citrate + | 320 | mg | 320 | mg | 320 | mg | 350 | mg | 350 | mg |
| | Magnesium Malate | | | | | | | | | | |
| Zinc | Zinc bisglycinate chelate | 10 | mg | 10 | mg | 15 | mg | 20 | mg | 20 | mg |
| Selenium | Selenomethionine | 200 | mcg | 200 | mcg | 200 | mcg | 200 | mcg | 200 | mcg |
| Chromium | Chromium picolinate | 100 | mcg | 30 | mcg | 35 | mcg | 45 | mcg | 45 | mcg |
| Potassium | Potassium citrate | 0 | mg | 0 | mg | 0 | mg | 45 | mg | 45 | mg |

Minerals (Separate pill for customization to benefit those with thyroid issues)

| | | | | | | | | | | | |
|---|---|---|---|---|---|---|---|---|---|---|---|
| Iodine | Potassium Iodide | 150 | mcg | 150 | mcg | 150 | mcg | 150 | mcg | 300 | mcg |

Fat Soluble

| Vitamin A (Beta Carotene) | Beta Carotene | 750 | mcg | 750 | mcg | 750 | mcg | 750 | mcg | 750 | mcg |
|---|---|---|---|---|---|---|---|---|---|---|---|
| Vitamin A (retinyl . . . ) | Retinyl palmitate | 300 | mcg | 300 | mcg | 300 | mcg | 400 | mcg | 500 | mcg |
| Vitamin D | Cholecalciferol | 100 | mcg | 50 | mcg | 50 | mcg | 75 | mcg | 100 | mcg |
| Vitamin E | Sunflower Oil | 335 | mg | 100 | mg | 100 | mg | 100 | mg | 335 | mg |
| Vitamin K1 | Phylloquinone | 50 | mcg | 50 | mcg | 50 | mcg | 60 | mcg | 70 | mcg |
| Vitamin K2 | Menaquinone-7 | 50 | mcg | 50 | mcg | 50 | mcg | 60 | mcg | 70 | mcg |
| Omega-3 (EPA) | EPA | 500 | mg | 500 | mg | 500 | mg | 500 | mg | 500 | mg |
| Omega-3 (DHA) | DHA | 500 | mg | 500 | mg | 500 | mg | 500 | mg | 500 | mg |
| Coenzyme Q10 | Ubiquinol | 200 | mg | — | | — | | — | | — | |
| Lutein | Lutein | 5 | mg | 5 | mg | 5 | mg | 7 | mg | 7 | mg |
| Zeaxanthin | Zeaxanthin | 2 | mg | 2 | mg | 2 | mg | 3 | mg | 3 | mg |

In addition to dividing the components set forth in Tables 2.1 and 2.2 between AM dosage form containers and PM dosage form containers for each stage of reproduction, each of these containers may include one or more solid oral dosage forms according to embodiments herein (e.g., tablets, capsules, sublinguals, etc.). In this Example, the water-soluble B vitamins listed in Table 2.1 were combined and formulated in one or more first dosage forms. The iron, Vitamin C and copper were combined and formulated in one or more second dosage forms. The first and second dosage forms were packaged together in the AM step with instructions directing a subject to administer the dosage forms in the morning (e.g., any time from about 6 AM to about 10 AM).

The PM dosage form packs included the Minerals listed in Table 2.2, which were combined together and formulated into one or more third dosage forms. The iodine was formulated in one or more fourth dosage forms. The iodine was formulated by itself into a separate dosage form to enable those with underactive thyroid issues to leave out the iodine component. For some people with an underactive thyroid, too much iodine can cause or worsen their condition. The fat-soluble components listed in Table 2.2 were combined and formulated together in one or more fifth dosage form. The third, fourth and fifth dosage forms were packaged together in the PM step and instructions directing a subject to administer the dosage forms in the evening (e.g., any time from about 4 PM to about 10 PM).

The preceding description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail or are presented in simple block diagram format in order to avoid unnecessarily obscuring the present invention. Thus, the specific details set forth are merely exemplary. Particular implementations may vary from these exemplary details and still be contemplated to be within the scope of the present invention.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the invention should be determined with reference to the appended claims, along with the full scope of equivalents to which such claims are entitled.

I/We claim:

1. A method of supplementing nutrition to a prenatal, pregnant or lactating woman, comprising providing to the woman:
one or more first dosage form, wherein the one or more first dosage form comprises at least one water-soluble compound;
one or more second dosage form, wherein the one or more second dosage form comprises iron and copper;
one or more third dosage form, wherein the one or more third dosage form comprises minerals;
optionally one or more fourth dosage form, wherein the one or more fourth dosage form comprises iodine; and
one or more fifth dosage form, wherein the fifth dosage form comprises at least one fat-soluble compound,
wherein the one or more first dosage form and the one or more fifth dosage form are formulated for administration at least four to twelve hours apart.

2. The method of claim 1, wherein at least one of the first to fifth dosage forms is free of folate, manganese or both.

3. The method of claim 1, wherein at least one of the first to fifth dosage forms comprises lutein.

4. The method of claim 1, wherein the at least one water-soluble compound comprises one or more of vitamin B1, vitamin B2, vitamin B3, vitamin B5, vitamin B6, vitamin B12, folate, folic acid, biotin, myo-inositol or D-chiro-inositol.

5. The method of claim 1, wherein the at least one water-soluble compound is free of one or more of a fat-soluble compound, iodine, a mineral, iron, vitamin C or copper.

6. The method of claim 1, wherein the one or more second dosage form further comprises vitamin C.

7. The method of claim 1 or 6, wherein the one or more second dosage form is free of one or more of a water-soluble compound other than vitamin C, a fat-soluble compound, iodine or a mineral other than iron and copper.

8. The method of claim 1, wherein the minerals comprise one or more of choline, calcium, magnesium, zinc, selenium, chromium or potassium.

9. The method of claim 1, wherein the one or more third dosage form is free of a water-soluble compound, a fat-soluble compound, iodine, iron and copper.

10. The method of claim 1, wherein the one or more fourth dosage form is free of a water-soluble compound other than iodine, a fat-soluble compound, a mineral, iron and copper.

11. The method of claim 1, wherein the at least one fat-soluble compound comprises one or more of one vitamin A, vitamin D, vitamin E, vitamin K1, vitamin K2, eicosapentaenoic acid, docosahexaenoic acid, coenzyme Q10, lutein or zeaxanthin.

12. The method of claim 1, wherein the one or more fifth dosage form is free of one or more of a water-soluble compound, iodine, a mineral, copper, iron or vitamin C.

13. The method of claim 1, wherein at least one of the one or more first dosage form, one or more second dosage form, one or more third dosage form and one or more fifth dosage form are comprised in a kit and the kit is provided to the woman.

14. The method of claim 1, wherein at least one of the first to fifth dosage forms or one or more sixth dosage form comprises a plurality of food nutrients selected from the group consisting of choline, citrus bioflavonoid complex, ginger, apple, banana, Spirulina, marshmallow, plum, a prebiotic, a probiotic, a digestive component, an enzyme component, bromelain, kiwi, papain, alpha linolenic acid, isomalto-oligosaccharides, milk thistle extract, Silybum marianum, silymarin, mixed tocopherols, marigold extract, Tagetes erecta, sunflower lecithin, date fruit, organic Murraya keonigii, Emblic Extract, Amla, Phyllanthus emblica, Sesbania grandiflora, Sea Kelp, Psidium guajava, Guava fruit, Lemon, Annatto, Ocimum sanctum, Holy Basil leaf, Mushroom, Agaricus bisporus, Moringa oleifera, Moringa leaf, Strawberry, Cherry, Blackberry, Blueberry, Raspberry, Beet, Broccoli, Carrot, Spinach, Tomato, Green Bell Pepper, Brussels Sprout, Ginger, Garlic, Green Onion, Parsley, Cauliflower, Red Cabbage, Asparagus, Celery, Cucumber, Kale, Spirulina, Marshmallow, Plum, Bromelain, Kwiki, Papain, red raspberry, Fubius idaeus, date fruit, derivatives thereof and combinations thereof.

15. The method of claim 1, wherein the one or more first dosage form is formulated for morning administration, the one or more second dosage form is formulated for morning administration, the one or more third dosage form is formulated for afternoon or evening administration, the one or more fourth dosage form is formulated for afternoon or evening administration and the one or more fifth dosage form is formulated for evening administration.

16. A kit, comprising:
one or more first dosage form, wherein the one or more first dosage form comprises at least one water-soluble compound;
one or more second dosage form, wherein the one or more second dosage form comprises iron and copper;
one or more third dosage form, wherein the third dosage form comprises minerals;
optionally one or more fourth dosage form, wherein the dosage form comprises iodine;
one or more fifth dosage form, wherein the fifth dosage form comprises at least one fat-soluble compound; and
instructions to administer the one or more first dosage form and the one or more fifth dosage form at least four to twelve hours apart.

17. The kit of claim 16, wherein at least one of the first to fifth dosage forms is free of folate, manganese or both.

18. The kit of claim 16, wherein at least one of the first to fifth dosage forms comprises lutein.

19. The kit of claim 16, wherein the at least one water-soluble compound comprises one or more of vitamin B1, vitamin B2, vitamin B3, vitamin B5, vitamin B6, vitamin B12, folate, folic acid, biotin, myo-inositol or D-chiro-inositol.

20. The kit of claim 16, wherein the at least one water-soluble compound is free of one or more of a fat-soluble compound, iodine, a mineral, iron, vitamin C or copper.

21-30. (canceled)
